(19) United States
(12) Patent Application Publication
(10) Pub. No.: US 2005/0096553 A1
(43) Pub. Date: May 5, 2005

(54) Electronic sphygmomanometer capable of timing measurement

(76) Inventor: Thi Ngoc Phuong Nguyen, Sales Le Chateau (FR)

Correspondence Address: HDSL, 4331 STEVENS BATTLE LANE, FAIRFAX, VA, 22033, US

(21) Appl. No.: 10/697,298

(22) Filed: Oct. 31, 2003

(51) Int. Cl. A61B 5/02

(52) U.S. Cl. 600/495; 600/493

(57) ABSTRACT

An electronic sphygmomanometer capable of timing measurement, having a key module, a controller, a memory, a manometer, a warning device and a display. The key module allows the user to input parameters such as the measuring time, interval, times, an abnormal threshold of blood pressure. The memory is controlled by the controller to store these parameters. According to a default time, a control signal is output from the controller to the manometer for performing measurement of blood pressure. Various measuring parameters and the measured blood pressure value are displayed by the display. When the measured blood pressure exceeds the threshold, a warning message is generated by the display and/or the warning device.

### BACKGROUND OF THE INVENTION

The present invention relates to an electronic sphygmomanometer and, more particularly, to an automatic sphygmomanometer controlled by a controller to perform timing measurement of blood pressure, and operative to generate a warning signal when the measured blood pressure is abnormal.

Blood pressure is an important indication for healthy condition. People gradually realize the importance of the blood pressure. As the living quality continuously enhances, high blood pressure has become a civilized disease that everybody is aware of and tries to prevent. Therefore, sphygmomanometer has become necessary health aid in every household.

The conventional sphygmomanometer includes the fully manual mechanism, which pumps air to increase pressure and requires the user listening to the pulse of the patient via an earpiece. This is very popular to medical professionals, but cannot be broadly applied to the public. Recently, electronic manometers have been very popular, which greatly improves the mechanical inconvenience. The electronic manometers can be divided into two major types, including fully automatic type and semi-automatic type. The former is operative to detect the arm of the patient wrapped within a ring and starts filling gas into the ring and measurement. This type has been used in medical institute and other public location. The former uses a touch button to activate gas filling and measurement, which is more applicable to personal and household use. For a patient with general high blood pressure or a healthy person, using this type of manometer to perform three measurements a day is enough. However, for a person with special illness (such as heart disease or diabetics) or a person under critical condition, timing periodic blood pressure measurements are required. For example, the measurement has to be performed every three minutes within 30 minutes, or the measurement is required every 20 minutes within certain period of time (such as between 22:00 and 7:00). The blood pressure variation of the patient can thus be intensively monitored. Under such circumstance, wither the above fully automatic or semi-automatic type of manometer cannot provide the timing measurement. Manual measurement is required. Should personal inadvertence or mistake occur, serious consequence may happen.

On the other hand, the conventional electronic manometer can only display the measured blood pressure value, but is not operative to generate a warning signal when the blood pressure value is abnormal. For a non-professional user, the abnormal blood pressure value may not mean anything. Therefore, even the correct blood pressure has been measured, a proper reaction or procedure will not be performed. Further, the blood pressure threshold value may not be the same for different individual, and the conventional manometer certainly cannot provides the specific threshold values for each individual.

### BRIEF SUMMARY OF THE INVENTION

The present invention provides an electronic sphygmomanometer capable of timing measurement. The user can input parameters such as time and number for measurement to automatically perform blood pressure measurement, such that man-made mistake or inadvertence can be avoided.

The present invention further provides an electronic sphygmomanometer capable for timing measurement, which allows the user to input blood pressure threshold value for each individual. When the measured blood pressure exceeds the threshold value, a warning signal can be generated to avoid the ignorance of user.

The electronic sphygmomanometer provided by the present invention comprises a controller as s control center, a key module and a display allowing the user to input parameters. According to the input parameters, the sphygmomanometer automatically performs timing measurement of blood pressure and display the measured result. When the measured result exceeds a pre-stored threshold value, a warning signal is generated.

### BRIEF DESCRIPTION OF THE DRAWINGS

These, as well as other features of the present invention, will become apparent upon reference to the drawings wherein:

FIG. 1 is a perspective view of an electronic sphygmomanometer according to the present invention; and

FIG. 2 is a block diagram of the electronic sphygmomanometer provided by the present invention.

### DETAILED DESCRIPTION OF THE INVENTION

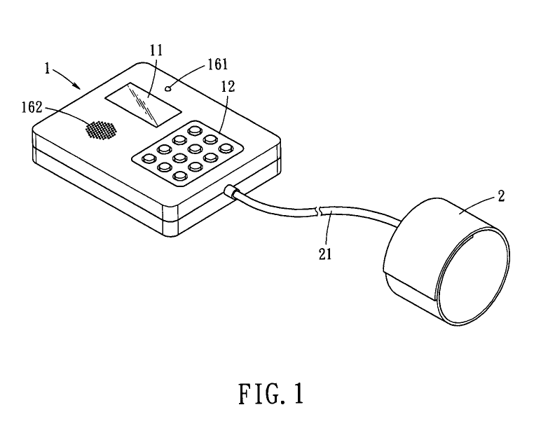

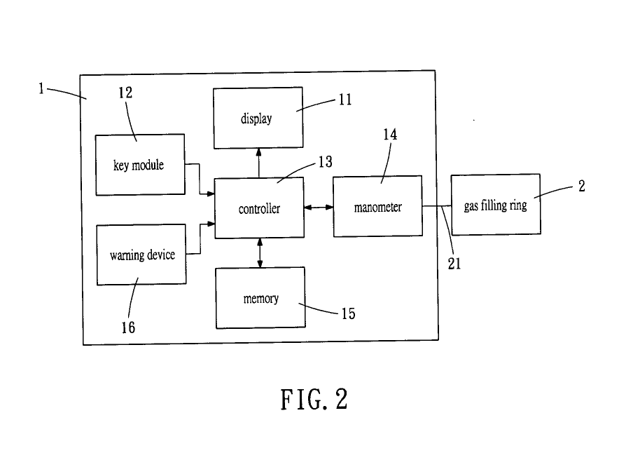

Referring to FIG. 1, a perspective view of an electronic sphygmomanometer provided by the present invention is illustrated. The sphygmomanometer comprises a bulk body 1 and a gas filling ring 2. The bulk body 1 and the gas filling ring 2 are connected to each other via a tube 21. The bulk body 1 comprises a display 11, a key module 12, a light emitting device 161, and an audio generator 162 formed on appropriate positions of the surface of the bulk body 1. The display 11 is operative to display information and input parameters, and the key module 12 allows the user to input the parameters. The display 11 includes liquid crystal display (LCD) or light emitting diode (LED) display for displaying digits or texts, for example. The light emitting device 161 and the audio generator 162 are used to generate a warning signal light and sound, respectively. The gas filling ring 2 is operative to apply pressure on the arm or wrist of the patient, which is an inevitable device for a manometer. FIG. 2 shows a block diagram of the present invention. As shown, the bulk body 1 further comprises a controller 13, a manometer 14, a memory 15 and a warning device 16 in addition to the display 11 and the key module 12. The manometer 14, the memory 15 and the warning device 16 are connected to the controller 13. The controller 13 is operative to receive the measuring time, interval, times, blood pressure threshold value input by the key module 12 and store these parameters into the memory 15. The manometer 14 includes an electronic manometer connected to the gas filling ring 2 via the tube 21. The manometer 14 is activated or inactivated by the controller 13. After the measurement, the manometer 14 is operative to transmit the measured result to the controller 13. When it is time for measurement as defaulted, a control signal is output by the controller 13 to the manometer 14, such that the gas filling ring 13 starts to pump gas therein. While discharging the gas within the ring 13, the blood pressure is measured, and the measured blood pressure value is transferred to the controller 13, which then compares the measured value to the threshold value stored in the memory 15. If the measured value exceeds the threshold value, a warning message is displayed on the display 11, and/or a warning light effect is generated by activating the warning device 16. The warning device 16 includes the light emitting device 161 and the audio generator 162 (as shown in FIG. 1) for generating a warning light or a warning sound, respectively. The light emitting device 161 includes a light emitting diode or a light bulb, and the audio generator 162 includes a speaker or a horn, for example. Thereby, a significant warning effect is obtained.

This disclosure provides exemplary embodiments of a child safety blind. The scope of this disclosure is not limited by these exemplary embodiments. Numerous variations, whether explicitly provided for by the specification or implied by the specification, such as variations in shape, structure, dimension, type of material or manufacturing process may be implemented by one of skill in the art in view of this disclosure.

1. An electronic sphygmomanometer capable for timing measurement, comprising:
a bulk body;
a key module installed on a surface of the bulk body, operative to input parameters including measuring time, measuring interval, measuring times, and abnormal blood pressure threshold value;
a display, mounted on the surface of the bulk body, operative to display information;
a controller, embedded in the bulk body;
an electronic manometer embedded in the bulk body and electrically connected to the controller, the electronic manometer being controlled by the controller to activate blood pressure measurement, the manometer being operative to transmit a measured blood pressure value to the controller;
a memory embedded in the bulk body and electrically connected to the controller, the memory being operative to store the parameters input by the key module; and
a gas filling ring connected to the manometer via a tube, the gas filling ring being operative to apply a pressure on an arm or a wrist of a user to perform blood pressure measurement on the user.

2. The electronic sphygmomanometer according to claim 1, wherein when it is time for measuring blood pressure of the user as input by the key module, the controller is operative to generate a control signal to the manometer, and to control the gas filling ring to fill gas therein, and when the measured blood pressure value transmitted from the manometer is higher than the threshold value, the display is operative to display a warning message.

3. The electronic sphygmomanometer according to claim 1, further comprising a warning device mounted inside or on the surface of the bulk body, the warning device is controlled by the controller to generate an audio and/or visual warning effect when the measured blood pressure value exceeds the threshold value.

4. The electronic sphygmomanometer according to claim 2, wherein the warning device includes a light emitting device operative to generate a warning light.

5. The electronic sphygmomanometer according to claim 3, wherein the light emitting device includes a light emitting diode or a light bulb.

6. The electronic sphygmomanometer according to claim 2, wherein the warning device includes a audio generator operative to generate a sound.

7. The electronic sphygmomanometer according to claim 6, wherein the audio generator includes a speaker or a horn.

8. The electronic sphygmomanometer according to claim 1, wherein the display includes a liquid crystal display or a light emitting diode display operative to display digits and/or texts.
